(19) United States
(12) Patent Application Publication
(10) Pub. No.: US 2010/0030223 A1
(43) Pub. Date: Feb. 4, 2010

(54) MODULAR PATELLA INSTRUMENT

(75) Inventor: Arnold Keller, Kayhude (DE)

Correspondence Address: MORRISON & FOERSTER LLP, 1650 TYSONS BOULEVARD, SUITE 400, MCLEAN, VA, 22102, US

(73) Assignee: WALDEMAR LINK GMBH & CO., KG, HAMBURG (DE)

(21) Appl. No.: 12/443,792

(22) PCT Filed: Aug. 27, 2007
(86) PCT No.: PCT/EP07/07492

§ 371 Date: Mar. 31, 2009

(30) Foreign Application Priority Data
Oct. 12, 2006 (EP) 06021418.6

(51) Int. Cl. A61B 17/58

(52) U.S. Cl. 606/99

(57) ABSTRACT

An instrument for implanting patellar prostheses in various sizes includes a series of milling calipers of various sizes, a retaining instrument with a receptacle for one of the milling calipers matching the sizes of the patellar prostheses and milling bodies in various sizes. The retaining instrument is configured like forceps with two members, the front end of one of the members being provided with a retention plate for engagement with a front face of a patellar prosthesis, and the front end of the other one of the members being provided with a holder for a milling gauge.

### REFERENCE TO RELATED APPLICATIONS

This application is the national stage under 35 USC 371 of International Application No. PCT/EP2007/007492, filed Aug. 27, 2007, which claims Convention priority from European Patent Application No. 06021418.6, filed Oct. 12, 2006, the full disclosures of which are incorporated herein by reference.

### FIELD OF THE INVENTION

The invention relates to a modular instrument set for implanting patella prostheses of various sizes, comprising a retaining instrument which is designed like forceps with two members, the front end of one member being provided with a retention plate for engagement with a front face of the patella, and the front end of the other member being provided with a guide for a milling gauge, and comprising several milling gauges which are available in various sizes matching the sizes of the patella prosthesis and are mounted exchangeably on the guide, and several milling bodies of various sizes, one each of which belongs to one of the milling gauges.

### BACKGROUND OF THE INVENTION

When a patient receives a knee prosthesis that ensures the articular function between femur and tibia, the implantation of a patella prosthesis is also generally indicated. Given the anatomy of the knee joint, the rear face of the patella bone cooperates with a front area of the femoral condyles during flexion of the knee. If, during implantation of a knee prosthesis, the natural condyles are replaced by condylar runners of the prosthesis, this causes contact between the natural patella and the condylar runners of the prosthesis. To avoid undesired contact between natural bone material and the artificial material of the prosthesis, generally of metal, an implant is provided on the rear face of the patella. This implant functions as an articulation surface and interacts with the corresponding mating surface on the condylar runners of the knee prosthesis. Because of this function, and because of the configuration of the natural patella, the patella prosthesis generally has a button-like shape. Its rear face acting as an articulation surface in most cases has a convex shape like a dome. Its front face for bearing on the natural patella bone is mostly flat, with optional anchoring pins. The implantation of the prosthesis involves bone material being removed by resection from the rear face of the natural patella bone, the thickness of the resected bone material corresponding approximately to the thickness of the patella prosthesis. This has the effect that functional joint anatomy can be preserved as far as possible. Resection of the patella bone is performed mainly by two techniques, namely sawing or milling.

An instrument set for sawing is known from prior public use (Columbus Knee System from Aesculap Orthopedics). Here, the thickness of the patella bone is determined, and the patella bone is squeezed into retaining forceps. These engage laterally and on the front face of the patella bone. In doing so, there is a danger of possible damage to tissue structures, in particular ligaments, which are likewise connected laterally to the patella. The retaining forceps include a saw guide. In this way, the operating surgeon is able to resect bone material from the rear face of the patella using an oscillating saw. The thickness of the bone material that is to be resected can be determined by means of a height adjustment device arranged on the retaining instrument. Resection by means of an oscillating saw has the effect that chips created by the sawing may pass into the surrounding area and thereby deposit themselves on other tissue material. Complete removal is often not possible. Leaving the chips permanently in situ poses a risk of infections or other complications. Moreover, the slit-shaped guide for the oscillating saw offers only inadequate guiding in the area of the point of the saw blade where it removes material. If the operating surgeon does not hold the instrument exactly at right angles to the slit in the saw guide, the oscillating saw removes either too much or too little material from the patella.

Instruments for milling the patella are also known, for example from U.S. Pat. No. 6,866,667. This includes a forceps-like instrument with a retention plate for supporting the front face of the patella on the front end of one member, and with a guide ring which is arranged on the front end of the other member and is designed to clamp the patella against the retention plate from the rear. The guide ring defines a zero position for a thickness measurement. The instrument further comprises an adjustable stop ring, which can be set to one of four different values depending on the size of the patella prosthesis intended for implantation. Depending on its setting, it permits a greater or lesser depth of insertion of a milling body through the guide ring, as a result of which bone material from the rear face of the patella is resected in accordance with the set thickness. It is the operating surgeon who sets the stop ring to the depth that is to be resected. This can lead to errors in setting. In particular, there is the danger of the stop ring being set to too great a depth, which depth belongs to a larger patella prosthesis than the one intended for implantation. Too much bone material would therefore be resected.

### SUMMARY OF THE INVENTION

The object of the invention is to improve an instrument set of the type mentioned at the outset, so as to avoid the abovementioned disadvantages and in particular to counter the risk of setting too great a thickness relative to the size of the patella prosthesis.

The solution according to the invention lies in an instrument set having the features of the invention as broadly described herein. Advantageous developments are the subject matter of the preferred embodiments.

In a modular instrument set for implanting patella prostheses of various sizes, comprising a retaining instrument which is designed like forceps with two members, the front end of one member being provided with a retention plate for engagement with a front face of the patella prosthesis, and the front end of the other member being provided with a holder for a milling gauge, and comprising several milling gauges which are available in various sizes matching the sizes of the patella prostheses and are mounted exchangeably on the holder, and several milling bodies of various sizes, one each of which belongs to one of the milling gauges, provision is made, according to the invention, that size gauges with a circumferential marker edge are provided whose sizes are in each case adapted to one of the milling bodies and which are integrated into the milling gauges, and several thickness rings are provided which can each be placed on the milling body of matching size and whose thickness corresponds to that of the patella prosthesis associated with the milling body.

Some of the terms used are explained below.

An instrument designed like forceps is to be understood as an instrument whose two members are connected to each other via a joint arranged in a central area of the members. This will in most cases be a pivot joint, although it is also possible to provide a joint for parallel guiding. At their rear end, the members have a grip part which is designed to be held with force by an operating surgeon.

Milling gauges are understood as elements which guide a milling body in at least one direction, preferably two directions, the milling bodies being movable in a third direction (mostly referred to as advance direction).

Patella prostheses of various sizes is to be understood as meaning that the patella prostheses have different widths. Although the patella prostheses do not form part of the instrument set according to the invention, their width nevertheless defines the width of the associated milling body. It is generally the case that the number of milling bodies of different diameters provided in the instrument set will correspond to the number of patella prostheses of various sizes that are intended to be implanted by means of the instrument set.

Thickness rings are understood as ring-shaped gauges that have a defined height. The height is the measure defined by the distance between the two end faces of the thickness ring.

The invention is based on the concept of integrating a size gauge into the milling gauge and of using this combined milling and size gauge to allow the operating surgeon to visually determine whether the selected milling gauge suits the patella bone that is to be worked on. The combined milling and size gauge means that the determination of the correct prosthesis size and the guiding of the resection work are brought together within a single component. The operating surgeon can select the largest prosthesis size in question and fit the corresponding milling gauge from the instrument set onto the retaining instrument. By means of the size gauge integrated into the milling gauge, the operating surgeon can check, by comparing with the circumferential marker edge, whether the patella is sufficiently large for the selected size. If the circumferential marker edge is covered completely by the patella, then the patella can accept this size; if there is no coverage at certain points, this then means that the patella is too small for the selected size. In the latter case, the next smaller size must then be selected, and the procedure repeated using the next smaller milling gauge and the size gauge integrated therein. Finally, when the correct size is determined by means of the size gauge integrated in the milling gauges, the diameter of the milling body to be used is also established. Since there is a specific thickness ring belonging to each milling body, and since only this fits onto the respective milling body, the thickness of the bone material to be resected from the patella is also automatically established.

The invention creates a seamless chain starting from the size gauge for selection of the correct prosthesis size and extending to the determination of the depth to which the milling body removes the bone material from the rear face of the patella. Therefore, with the modular instrument set according to the invention, neither measurement nor adjustment is necessary. By virtue of the invention, errors resulting from incorrect adjustment can no longer arise. Handling is thus made easier and also more reliable.

In addition to the thickness ring, a displaceable stop ring is also expediently provided. It serves to form a depth stop directly on the milling body. In this way, a depth stop does not have to be provided on the retaining instrument. The arrangement of the stop ring directly on the milling body also has the advantage that imprecision in the positioning of the retaining instrument does not have any effect on the depth determination. To make adjustment of the stop ring easier, a clamping device is preferably provided on the stop ring. It permits simple positioning and securing of the stop ring in the desired position relative to the milling body.

The stop ring is preferably designed congruent to the thickness ring. This ensures a large contact area between the end faces of stop ring and thickness ring that come to bear on each other. The congruent design also ensures easier handling. It is clear by intuition to the operating surgeon that the thickness ring has to be fitted onto the milling body (and of course it only fits onto one according to the invention) in such a way that it bears on the stop ring. This further counters the risk of incorrect handling.

The retaining instrument designed like forceps expediently has a joint which is designed for linear guiding of the members. This is achieved by the retention plate on one member and the milling gauge on the other member moving linearly along an axis to each other, but otherwise not changing their orientation with respect to each other. This avoids the angle differences that would arise in the case of guiding along an arc of a circle in a simple pivot joint. This permits more precise guiding and, as a result, better protects the areas of the patella and adjoining ligaments that come into contact with the instrument according to the invention.

Drill jigs of various sizes are expediently provided. They allow bores to be drilled into the milled rear face of the patella. These bores serve to receive anchoring pins of the patella prosthesis. In order to maintain a defined angle position, the drill jigs are expediently provided with an angle marking. It is particularly expedient if the latter is designed as a locking means, for example in the form of a pin which, in the correct position, engages in a matching recess on the milling gauge. In this way, the angle position is securely fixed.

A pressing device is preferably provided that can be mounted on the holder. It is used for pressing the patella prosthesis onto the implantation site after the rear face of the patella has been resected and, if appropriate, after the bores have been drilled. The arrangement of the pressing device on the retaining instrument according to the invention has the advantage that the retaining instrument does not have to be removed when pressing the prosthesis in. This has advantages in cases where subsequent working proves necessary.

A drill fixture is preferably provided which has a drill limited by a depth stop. This can be integrated into the milling gauge instead of the milling body. This may be expedient if, in the case of a very hard patella bone, the hard surface first has to be broken before the actual milling is carried out by means of the milling body. The depth stop on the drill ensures that the drill does not penetrate too deep and adversely affect the planicity of the surface intended to receive the patella prosthesis. It generally suffices if the depth stop is selected such that a sufficient distance remains in the case of the smallest size of patella prosthesis. If so desired, however, stops with different depths or displaceable stops can be provided in order to permit, for the larger patella sizes, a deeper breaking by means of the drill.

According to a particularly expedient embodiment that is possibly deserving of independent protection, a saw jig module is provided which can be mounted on the holder of the retaining instrument. With the saw jig module, it is possible to resect the patella bone by sawing instead of by milling. This is advantageous especially if it transpires, during surgery, that resection with a milling device is inappropriate. It is then possible, without having to exchange the instrument set, to perform the resection by means of the saw jig module. For this purpose, guides are expediently provided on the size gauge module. They are of such a nature that the saw jig module can be safely mounted in a defined position on the size gauge module. The guide is preferably designed as a double guide, such that the saw jig module can be arranged on two sides of the size jig module. This allows the operating surgeon to choose the optimal position of the saw jig module according to the individual access conditions.

The guide on the size gauge module is preferably designed such that the saw jig module is guided in a vertically adjustable manner on the size gauge module. In this way, the thickness of the material to be removed from the patella bone can be determined by a displacement of the saw jig module relative to the size gauge module.

In order to arrange the saw jig module in the correct position on the size gauge module, a depth-measuring device is expediently provided. It is designed to probe the thickness of the natural patella bone and, on the basis of this, to determine the height of the saw jig module such that, during sawing, bone material is removed to a thickness that corresponds to the thickness of the patella prosthesis to be implanted. For this purpose, the depth-measuring device has a probe which protrudes down into the opening formed by the size gauge. This probe is expediently designed as a probe star, with a plurality of probe bodies arranged in a radiating pattern. The probe bodies differ in terms of their dimensions, with each probe body being assigned to one size of the patella prostheses. The extent of the probe bodies in the radial direction corresponds to the thickness of the respective patella prosthesis. By rotating the star such that the probe body for the size intended for implantation points in the direction of the patella retention plate, the depth-measuring device is correctly adjusted automatically, without any further manual maneuvers. The probe device then simply has to be moved together with the saw jig module along the guide, until the probe device bears on the rear face of the patella bone. In this way, the position of the saw jig module is determined, and thus the plane for the bone resection by means of a saw.

### BRIEF DESCRIPTION OF THE DRAWINGS

The invention is explained below with reference to the attached drawing, which depicts an advantageous illustrative embodiment of the invention and in which:

FIG. 1 shows a view of an illustrative embodiment of the instrument according to the invention;

FIG. 2 shows a detail of the instrument from FIG. 1;

FIG. 3 shows size gauges of the instrument set to which the instrument according to FIG. 1 belongs;

FIG. 4 shows milling bodies of the instrument set in two different sizes;

FIG. 5 shows thickness rings of different sizes and a milling body;

FIG. 6 shows drill jigs of the instrument set according to the invention;

FIG. 7 shows a patella prosthesis for implantation using the instrument set;

FIG. 8 shows the determination of a correct prosthesis size using the instrument set according to the invention;

FIG. 9 shows the insertion of a milling body for determination of a starting position;

FIG. 10 shows the patella being milled;

FIG. 11 shows a tip of the patella being broken;

FIG. 12 shows the patella prosthesis being pressed in;

FIG. 13 shows a perspective view of a saw jig for the instrument set according to the invention; and

FIG. 14 shows the instrument set with the saw jig mounted on the patella.

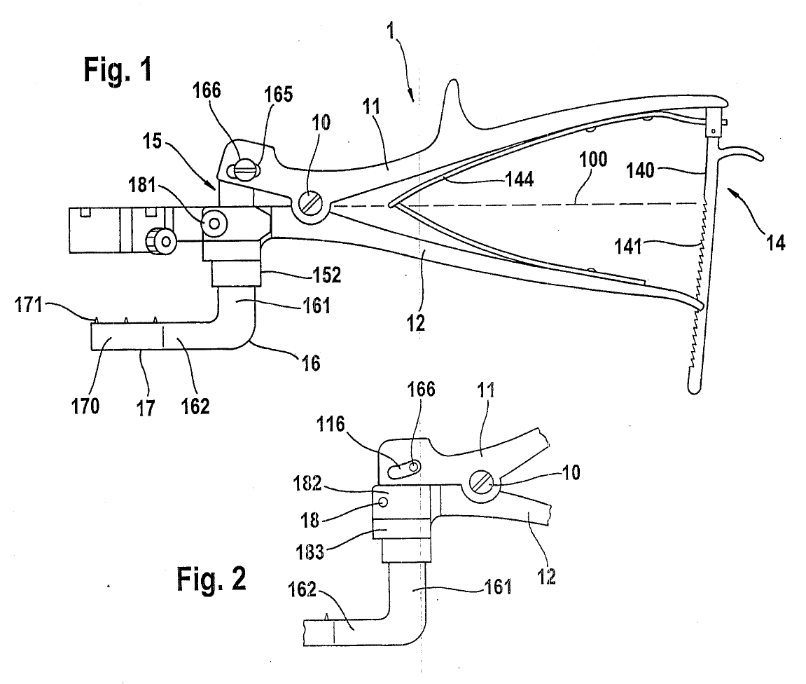

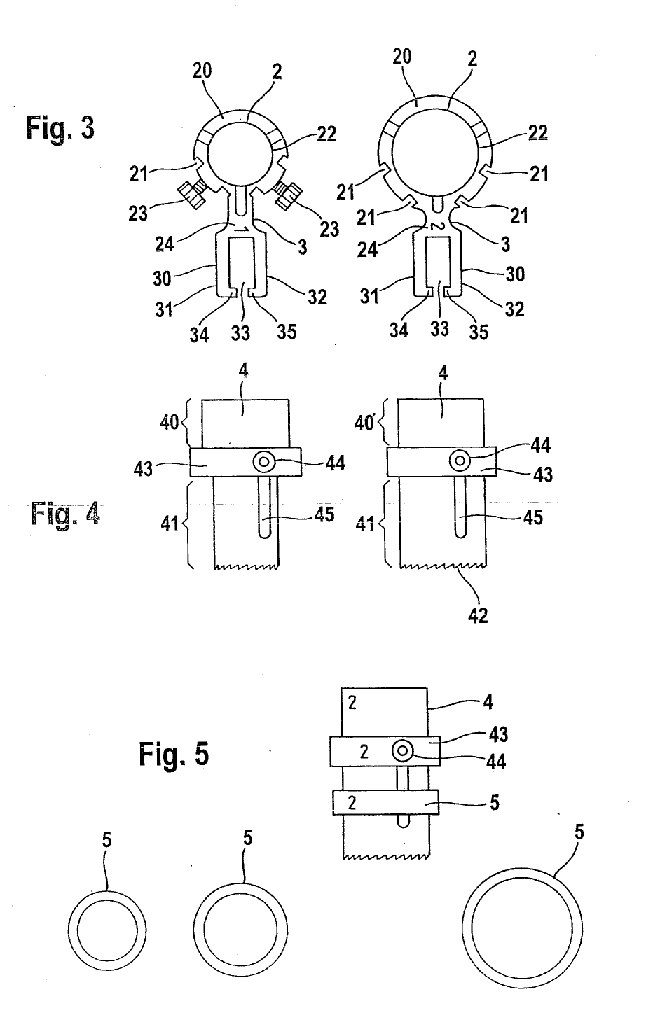

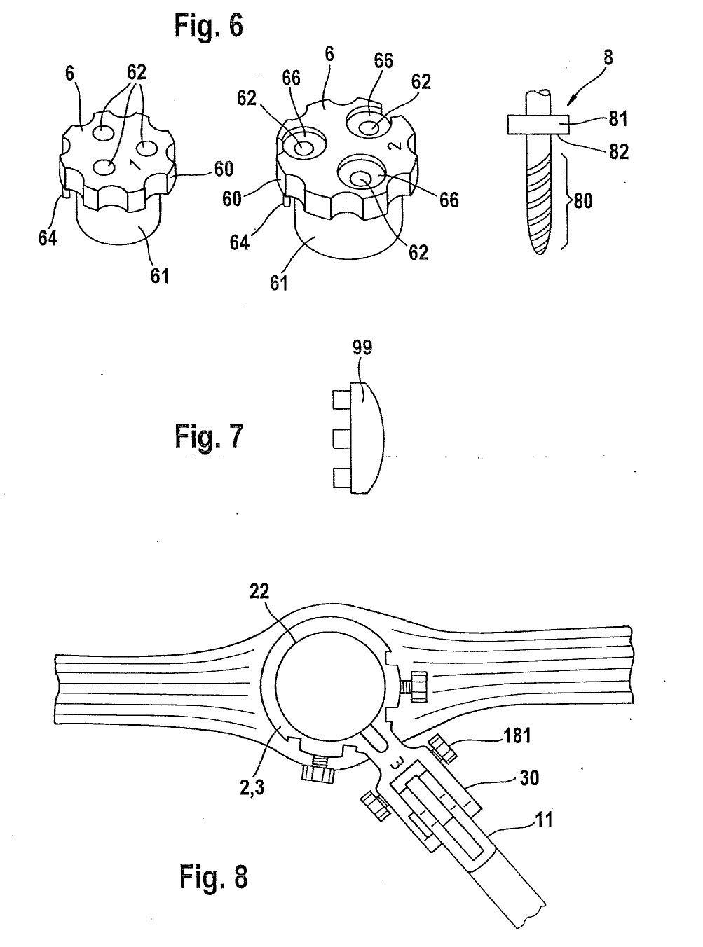

### DETAILED DESCRIPTION OF THE INVENTION

An instrument set according to the invention comprises a retaining instrument 1, a plurality of milling gauges 3, each of them with an integrated size gauge 2, a corresponding number of milling bodies 4 adapted in size to the milling and size gauges 2, 3, thickness rings 5 assigned in each case to one of the milling bodies 4, a corresponding number of drill jigs 6 and pressing-in devices 7, a drill 8, and a saw jig module 9.

Patella prostheses are provided in different sizes for implantation. One example is shown in FIG. 7. The patella prosthesis 99 comprises a support body with a convex front face and several pegs on the rear face.

The retaining instrument 1 is designed like forceps, with two members 11, 12 arranged movably about a pivot joint 10. Both members are designed as grips in their rear section, and at the rear end there is a locking device 14 for fixing the retaining instrument 1 in a clamped position. The locking device 14 comprises a rod 140 which is arranged pivotably on the member 11 and which has saw teeth 141 on its front face. This engages in a corresponding edge (not shown) of a recess at the rear tip of the member 12. A V-shaped leaf spring 144 is provided for applying a force that drives the members 11, 12 apart.

The front end of the members 11, 12 is designed as a linear guide 15. It comprises a guide sleeve 152 in the front end of the member 12, in which a branch 161 of an L-shaped retention bracket 16 is guided longitudinally displaceably in a transverse axis of the instrument 1. Transverse axis is understood here as an axis that lies perpendicular to the longitudinal axis 100 of the retaining instrument 1, which extends from front to rear through the pivot joint 10. The other branch 162 of the retention bracket 16 extends forward parallel to the longitudinal axis 100 and has a retention plate 17 at its front end. This has an annular edge 170, with a plurality of spikes 171 distributed uniformly thereon and pointing toward the main axis 100 of the instrument. The retention plate 17 is designed to grip and fix the rear aspect of the patellar tendon when the retaining instrument 1 is fitted in place. The end of the branch 161 remote from the retention plate 17 is connected to the front end of the member 11 via a pivot bearing 165. The pivot bearing 165, in its simplest form, can be designed as a curved slit 116 at the front end of the member 11, the shaft being guided on a screw 166. The curvature and orientation of the slit 116 are chosen such that, in addition to the pivoting movement, length compensation is also achieved. A multi-purpose holder 18 is formed at the front end of the member 12 along with the guide sleeve 152. It serves to ensure that the size and milling gauges 2, 3, the pressing module 7 and the saw jig module 8 are mounted exactly in position on the instrument 1. The multi-purpose holder 18 has a stepped configuration and comprises an upper section directed toward the member 11, the material thickness decreasing in the direction of the center axis 100. This section is designated as thinner section 182, while a section adjoining this in the direction of the member 12 is designated as thicker section 183.

In order to protect against accidental loosening or displacement, a locking screw 181 is provided on the respective insert or module.

The size and milling gauges 2, 3 are provided in different sizes, of which FIG. 3 shows two from a set of, for example, four in total. They are composed principally of an annular sleeve 20 and of a retainer clip 30 that extends radially outward from the latter. The retainer clip 30 has two retaining branches 31, 32 that border the U-shaped space 33 for receiving the multi-purpose holder 18 with an exact fit. Moreover, arranged at the outer ends of the branches 31, 32, there are mutually facing projections 34, 35 that enclose the multi-purpose holder 18 in the assembled state. A seat for the locking screw 181 is arranged on at least one of the branches. The width between the projections 34, 35 is dimensioned such that it is sufficient to permit passage of the thinner section of the multi-purpose holder 18, but prevents passage of the thicker section. On its periphery, the annular sleeve 20 has two pairs of guide grooves 21 which are arranged at an angle to each other and extend in the axial direction along the full height of the annular sleeve 20. A clamping screw 23 is arranged in a thread between the two guide grooves 21 of each pair. This device is used for securing additional modules, as will be explained in more detail below. On the top surface of a transition area between the annular sleeve 20 and the retaining branch 30 there is a distinctive size marking 24 (for example the numbers 1, 2, 3 or 4) which clearly identifies the respective dimension of the size and milling gauge 2, 3. A particularly important role is played by the circumferential inner lower edge 22 of the annular sleeve 20. It is designed as a marker edge. This means that its contour is designed to exactly match the outer contour of the patella prosthesis 99. This means that the marker edge 22 exactly delimits the area on which the patella prosthesis 99 is to be implanted. For each size of the patella prostheses 99, there is a size and milling gauge 2, 3 whose marker edge 22 exactly follows the outer contour of the respective patella prosthesis 99. This ensures a clear association between the respective size and milling gauge 2, 3 and a size of the patella prostheses 99. This allows the operating surgeon to make a simple and reliable choice of the correct size of patella prosthesis 99 and, at the same time, allows him to safely establish the elements of the instrument set according to the invention that are to be used to implant these sizes. Preferably starting with the largest size, the operating surgeon holds the size gauges 2 in succession over the patella bone on which the patella prosthesis 99 is to be implanted. It is then simply necessary to check whether or not the marker edge 22 is completely covered by the patella bone. If this is not the case, the patella bone is too small for the size being checked, and the next smaller size is then to be chosen, with this procedure being repeated until finally the size gauge 2 is found in which the marker edge 22 is covered completely by the patella bone. In this way, the correct size of the patella prosthesis is found. This size gauge 2 can now be secured on the retaining instrument 1 by means of the multi-purpose holder 18. Since, according to the invention, the size gauge 2 is at the same time also the milling gauge 3, to which only one milling body 4 precisely fits, this therefore ensures at the same time that the correct milling gauge is used and thus also the correct milling body 4. This eliminates errors that are caused by accidentally using too large a milling body 4 and, consequently, by removing the wrong amount of bone material irretrievably.

When the size and milling gauge 2, 3 of the correctly defined dimension is fixed on the retaining instrument 1, the rear face of the patella bone can be prepared for implantation of the patella prosthesis 99 of the selected size. In cases where there are hard and very osseous rear surfaces of the patella bone, provision can optionally be made to use the drill 8 to break the tip on the rear face of the patella and thus make access easier for the milling bodies 4. At its front section, the drill 8 has a helical area 80 which is designed in the normal manner for a drill. This area is adjoined to the rear by a collar 81. The face 82 of the latter directed toward the helical section 80 functions as an abutment surface against a top face of an abutment plate 86. The abutment plate 86 is designed as a circular disk and lies like a cover on the annular sleeve 20 of the selected size and milling gauge 2, 3. It has a central opening which is dimensioned for passage of the helical section 80 of the drill but is smaller than the diameter of the collar 81. In this way, during drilling, the drill 8 can engage in the annular sleeve 20 only to a depth at which the collar 81 bears with its face 82 on the plate 86. The height of the annular sleeve 20 is chosen as a function of the dimension of the respective size and milling gauge 2, 3 such that the drill 8, on reaching the abutment, only removes the upper tip area of the patella bone. This ensures that the drill 8 does not remove bone material beyond the actual implantation depth.

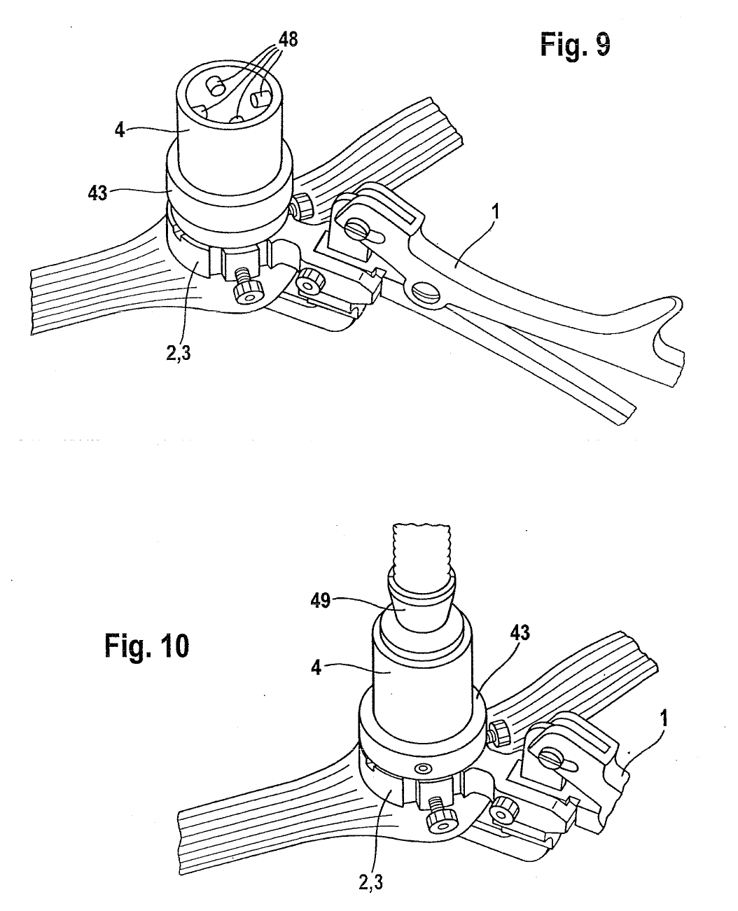

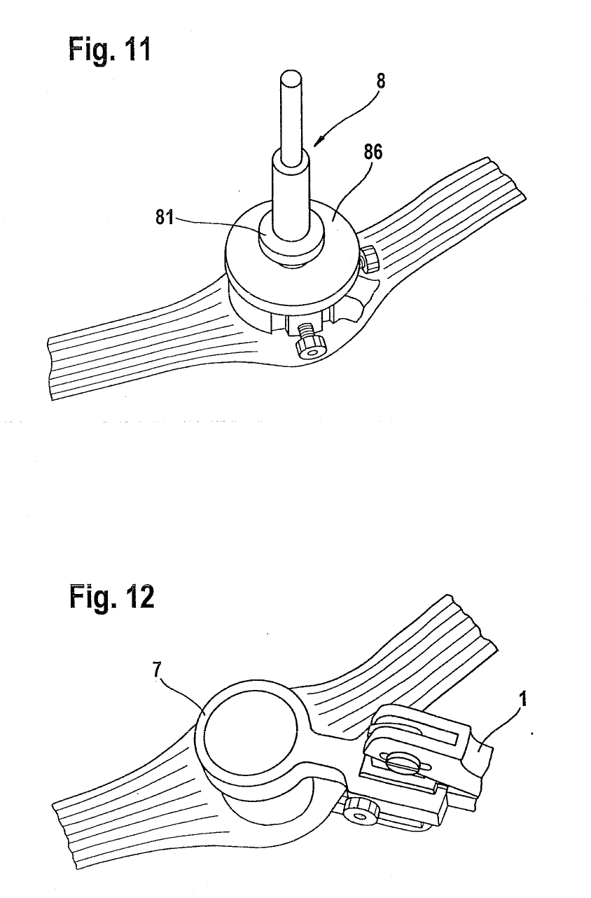

The four differently dimensioned milling bodies 4 in the depicted illustrative embodiment each have an upper section 40 and a lower section 41. The upper section 40 is designed like a sleeve and has, on its inside 4, radially inwardly directed projections 48 (see FIG. 9) as coupling elements for a drive tool 48 (see FIG. 10). The milling body 4 is driven via these coupling elements. The upper section 40 has the same width in all the milling bodies 4. The lower section 41 has a cylinder shape with milling teeth 42 on its bottom end face. The diameter of the lower section 41 differs depending on the dimension of the respective milling body 4. It corresponds in each case to the diameter of the different sizes of patella prosthesis 99. Apart from a slight play that is provided to avoid jamming, this diameter corresponds to the internal diameter of the annular sleeve 20 of the respectively associated size and milling gauge 2, 3. A stop ring 43 is pushed onto the lower section 41. It is guided longitudinally displaceably, with the aid of a clamping screw, in an axial groove 45 on the jacket of the lower area 41. The stop ring can be pushed to any desired position along the groove 45 and can be fixed there by means of the clamping screw 44. The internal diameter of the stop ring 43 is chosen such that it sits practically free of play on the lower section 41; its external diameter is chosen so as to coincide with that of the annular sleeve 20 of the associated size and milling gauge 2, 3. For milling, the milling head 4 is inserted into the previously selected size and milling gauge 2, 3 until its end face with the milling teeth 42 bears on the tip of the rear face of the patella bone. From this position, the depth of bone removal is to be determined that is equal to the thickness of the support element of the patella prosthesis 99. The milling body 4 is intended to remove material to a thickness corresponding to the thickness of the support body of the patella prosthesis 99.

A respective thickness ring 5 is provided for each size of patella prosthesis 99. Its height corresponds in each case to the thickness of the support body of the patella prosthesis 99, for which reason the height is also designated as “thickness”. The internal diameter of the inner rings 5 is in each case chosen so as to coincide with the external diameter of the lower area 41 of the respective milling body 4 belonging to the respective size of patella prosthesis 99. This ensures a clear and unmistakable allocation between thickness rings 5 and milling bodies 4 with respect to the respective size of patella prosthesis 99. Provision is made that the thickness ring 5 assigned to the selected milling body 4 is pushed from underneath onto the lower section 41 and is brought into contact with the stop ring 43. The selected milling body 4, with the thickness ring 5 pushed on, is then inserted into the allocated size and milling gauge 2, 3 and pushed forward until the end face of the lower section 41 with the milling teeth 42 bears on the tip of the rear face of the patella bone. The stop ring 43 is then moved down toward the annular sleeve 20 until the thickness ring 5 abuts against the top surface of the annular ring 20. In this position, the clamping screw 44 of the stop ring 43 is tightened. The depth of the milling to be carried out with the milling body 4 is thus determined from the zero point defined by the height of the tip of the patella bone and from the thickness, as represented by the thickness ring 5, of the patella prosthesis that is to be implanted. The stop ring 43 thus functions now as a depth stop. The milling body 4 is detached from the milling and size gauge in order to remove the thickness ring. It is then fitted back in place and can be connected to the drive tool. The milling can then be carried out until the milling body 4 has been advanced to the extent that the stop ring 43 bears on the top surface of the annular sleeve 20 (see FIG. 9). The maximum milling depth corresponding to the thickness of the selected size of patella prosthesis 99 is reached in this way. Errors of the kind that could arise by accidentally milling too deeply are reliably ruled out in this way.

The milling body 4 can now be removed, and bores for securing purposes can be provided by means of the drill jigs 6. The drill jigs 6 are of a mushroom-like shape, with a lower section 61 whose diameter is adapted to the internal diameter of the annular sleeve 20 of the respective size and milling gauge 2, 3. An upper section 60 projects like a collar and has a diameter corresponding to the external diameter of the annular sleeve 20. Three through-bores 62, offset by 120°, are arranged on the top surface of the drill jig 6 and extend through the upper section 60 under the lower section 61. Depending on the dimension of the drill jig, a countersink 66 can be provided surrounding the respective bore 62. It is designed to function as a depth stop for a drill engaged through the bores 62. This ensures that the drill can reach a predefinable depth relative to the underside of the upper section 60 functioning as reference surface. Drilling the securing holes for the pegs of the patella prosthesis 99 too deep can be easily and reliably avoided in this way. Arranged on the reference surface there is a pin 64, which engages in a corresponding recess on the size gauge/milling gauge 2, 3 and functions as an angle marking.

With the removal of the drill jig 6, the patella bone is ready for implantation of the patella prosthesis 99 of the selected size. The thickness of the removed bone material corresponds exactly to the thickness of the support element of the patella prosthesis 99. The depth of the holes corresponds to the length of the securing pegs 96 of the patella prosthesis 99. In contrast to conventional instrument sets, there is no danger of the milling or drilling being too deep. By virtue of the instrument set according to the invention, it is possible instead to ensure that the thickness of the removed bone material corresponds exactly to the size of the patella prosthesis 99, such that a more reliable and anatomically correct positioning of the patella prosthesis 99 is achieved.

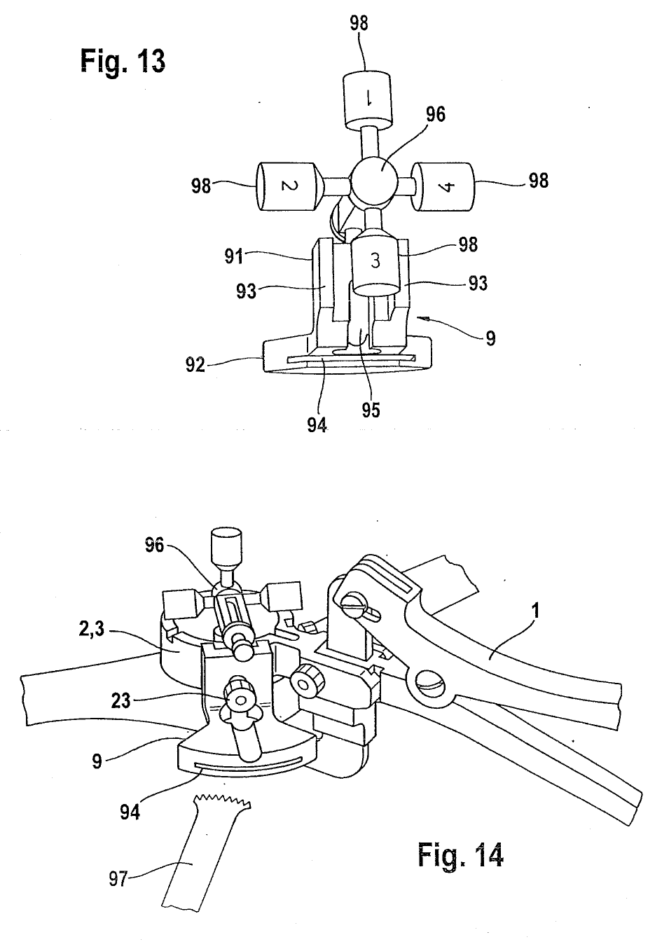

FIGS. 13 and 14 show the saw jig module 9, which is provided for carrying out the alternative resection method of sawing instead of milling. The saw jig module 9 has an L-shaped main body with a base branch 91 and a guide branch 92. On its side facing away from the guide branch 92, the base branch 91 has guide ledges 93 which are designed for form-fit engagement in the guide grooves 21 of the combined milling/size gauge 2, 3. They permit a height-adjustable arrangement of the saw jig module 9 on the milling/size gauge 2, 3. A slit 95 is arranged extending parallel between the guide ledges 93. The clamping screw 23 can be engaged through this slit 95 in order, when tightened, to lock the saw jig module 9 in its position. The guide branch 92 has a guide slit 94 which defines a cutting plane for a saw blade 97. The cutting plane is oriented here such that its normal vector extends in the direction of the guide ledges 93, with the result that, when the saw jig module is assembled, the cutting plane lies parallel to the upper edge of the retention plate 170.

Arranged on the base branch 91, on the end directed away from the guide branch 92, there is a probe star 96. It has four probe bodies 98 which are offset by 90° and which point in the radial direction. They each have a different length, with one of the probe bodies 98 being assigned in each case to a size of the prosthesis 99. The different lengths reproduce the different thicknesses of the prostheses 99 in the different sizes. The probe star 96 is arranged to be rotatable, such that one of the probe bodies 98 can be brought in each case into a position pointing in the direction of the guide slit 94. This probe body is the active probe body. Its identification corresponds to that of the used size/milling gauge module 2, 3.

During the operation, the surgeon simply has to move the saw jig module 9 down along its guide formed by the guide ledges 93 running in the guide grooves 21, until the probe body 98 belonging to the size/milling gauge 2, 3 to be used bears with its outer surface on the top of the rear face of the patella bone that is to be resected. The correct position of the saw jig module 9 is found in this way. The clamping screw 23 can be fixed. The guide slit 94 for the saw blade 97 is then located at the correct height, namely at a distance, corresponding to the thickness of the patella prosthesis 99, below the highest point of the patella bone. The resection by sawing can now be carried out in a simple way by inserting the saw blade 97 into the guide slit 94 and performing the sawing action. The instrument with the saw jig module 9 according to the invention does not have to be withdrawn, and instead the sawing can be performed directly in one procedure.

The guide grooves 21 are arranged on the size/milling gauge 2, 3 in such a way that the saw jig module 9 protrudes rearward on both sides at a 45° angle with respect to the longitudinal axis 100 of the instrument. This arrangement represents the best possible compromise between accessibility and favorable guiding of the saw blade. Therefore, with the saw jig module 9 according to the invention, the sawing can be carried out easily and accurately, and the possibilities of error are reduced to a minimum.

1-14. (canceled)

15. A modular instrument set for implanting patellar prostheses of various sizes, comprising:
a retaining instrument configured like forceps with two members, a front end of a first member being comprising a retention plate for engagement with a front face of a patellar prosthesis, and a front end of a second member comprising a holder for a milling gauge,
a plurality of milling gauges of various sizes matching the sizes of the patellar prostheses mounted exchangeably on the holder,
a plurality of milling bodies of various sizes, each one of which corresponds to one of the milling gauges,
a module of size gauges with circumferential marker edges, each of which is of a size adapted to a corresponding one of the milling bodies and which are integrated into corresponding milling gauges, and
a plurality of thickness rings which are configured to be placed on a milling body of matching size and whose thickness corresponds to that of the patellar prosthesis corresponding to the milling body.

16. The modular instrument set of claim 15, further comprising a displaceable stop ring provided on one of the milling bodies.

17. The modular instrument set of claim 16, further comprising a clamp provided on the stop ring.

18. The modular instrument set of claim 16 or 17, wherein the stop ring is congruent to the thickness ring.

19. The modular instrument set of claim 15 or 16, wherein the instrument has a joint which is designed for linear guiding of the members.

20. The modular instrument set of claim 15 or 16, further comprising drill jigs of sizes corresponding to the patellar prostheses.

21. The modular instrument set of claim 20, wherein the drill jigs are provided with an angle marking.

22. The modular instrument set of claim 15 or 16, further comprising a pressing device configured to be mounted on the holder.

23. The modular instrument set of claim 15 or 16, further comprising a drill fixture which has a drill limited by a depth stop.

24. The modular instrument set of claim 15 or 16, further comprising a saw jig module configured to be mounted on the holder of the retaining instrument.

25. The modular instrument set of claim 24, further comprising guides for the saw jig module provided on the size gauge module.

26. The modular instrument set of claim 24, further comprising a depth-measuring device provided on the saw jig module.

27. The modular instrument set of claim 26, wherein the depth-measuring device has a probe that protrudes downward into an opening formed by the size gauge.

28. The modular instrument set of claim 27, wherein the probe is in the form of a probe star having a plurality of probe bodies that are arranged in a radiating pattern and are each of a different size.
